(19) United States
(12) Patent Application Publication
(10) Pub. No.: US 2025/0280855 A1
(43) Pub. Date: Sep. 11, 2025

(54) A FEED COMPOSITION AND USE THEREOF

(71) Applicant: DSM IP ASSETS B.V., Heerlen (NL)

(72) Inventors: Wen REN, Wuhan (CN); Sébastien Eric POTOT, Blotzheim (FR); Maria WALSH, Kilkenny (IE)

(21) Appl. No.: 18/729,670

(22) PCT Filed: Feb. 9, 2022
(86) PCT No.: PCT/CN2022/075630

§ 371 Date: Jul. 17, 2024

(51) Int. Cl. A23K 20/111; A23K 20/132; A23K 50/10; A23K 50/20; A23K 50/30; A23K 50/75

(52) U.S. Cl. CPC A23K 20/111; A23K 20/132; A23K 50/10; A23K 50/20; A23K 50/30; A23K 50/75

(57) ABSTRACT

A composition comprising benzoic acid, derivatives or metabolites thereof, in combination with at least one essential oil selected from the group consisting of thymol, eugenol and piperine, for improving growth performance of an animal infected with Streptococcus suis.

### CROSS-REFERENCE TO RELATED APPLICATIONS

This application is the U.S. national phase of International Application No. PCT/CN2022/075630 filed Feb. 9, 2022, which designated the U.S., the entire contents of which are hereby incorporated by reference.

### TECHNICAL FIELD

The present invention relates to a feed composition for improving growth performance of animals infected with Streptococcus suis.

### BACKGROUND OF THE INVENTION

Streptococcus suis is a gram-positive bacterium that is a major porcine pathogen worldwide. The organism is carried in the tonsils of pigs and spreads between animals mainly by nose-to-nose contact or by aerosol over short distances. Streptococcus suis causes meningitis, septicaemia, endocarditis, arthritis and septic shock with high mortality.

Humans can be infected with Streptococcus suis when they handle infected pig carcasses or meat, especially with exposed cuts and abrasions on their hands. Human infection can be severe, with meningitis, septicaemia, endocarditis and deafness as possible outcomes of infection.

While antibiotic treatment of the infection of Streptococcus suis can be effective, there is a continuing drive to reduce the reliance on antibiotics in intensive production facilities, and as such, alternatives to antibiotics are being investigated.

Due to increasing concern that antibiotic-resistant strains of Streptococcus suis are entering the meat supply chain to infect humans, and the background of continuous reduction of antibiotic use in animals, there is a need to explore non-antibiotic approaches to controlling Streptococcus suis in animals.

It is known from EP2042041 that benzoic acid, derivatives or metabolites thereof in combination with a mixture of essential oils can be used to improve feed conversion ratio and/or daily weight gain and/or modulate gut flora in animals.

It has now been found surprisingly that in addition to the above function, benzoic acid, derivatives or metabolites thereof, in combination with a mixture of essential oils has the advantage of being able to improve growth performance of an animal infected with Streptococcus suis.

### SUMMARY OF THE INVENTION

Accordingly, the present invention provides a composition comprising benzoic acid, derivatives or metabolites thereof, in combination with at least one essential oil for improving growth performance of an animal infected with Streptococcus suis.

### DESCRIPTION OF THE FIGURES

FIG. 1 shows a summary of the mean bodyweight gain per piglet in each group.

FIG. 2 shows a summary of the daily mean feed consumption per piglet in each group.

### DETAILED DESCRIPTION OF THE INVENTION

Particularly, the present invention relates to a feed composition comprising benzoic acid, derivatives or metabolites thereof, in combination with at least one, preferably at least two, more preferably at least three essential oils selected from the group consisting of thymol, eugenol and piperine, for improving growth performance of an animal infected with Streptococcus suis.

The present invention also relates to the novel use of the feed composition as defined above for improving growth performance of an animal infected with Streptococcus suis.

The invention further relates to a method for improving growth performance of an animal infected with Streptococcus suis, comprising administering to the animal an effective amount of the feed composition as defined above.

In the present invention, improvement of the growth performance can be represented by the increase of bodyweight gain (WG) and/or increase of feed intake (FI) of the animals with supplementation of benzoic acid or derivatives or metabolites thereof, in combination with at least one essential oil selected from the group consisting of thymol, eugenol and piperine, compared to the animals without supplementation of the combination.

Benzoic acid is commercially available (for example as VevoVitall®, supplied by DSM Nutritional Products, Switzerland) or can easily be prepared by a skilled person using processes and methods well-known in the prior art.

Specific examples of derivatives of benzoic acid which can give rise to benzoic acid in vivo include salts of benzoic acid such as the alkali metal salts, alkaline earth metal salts and ammonium benzoates.

The essential oil compounds according to the invention are commercially available or can easily be prepared by a skilled person using processes and methods well-known in the prior art.

The essential oil compounds can be used in highly purified forms in mixtures or in the form of natural available plant extracts or extract-mixtures.

The term “extract” as used herein includes compositions obtained by solvent extraction (which are also known as “extracted oils”), steam distillation (which are also known as “essential oils”) or other methods known to the skilled person. Suitable extraction solvents include alcohols such as ethanol.

By the expression “natural” is in this context understood a substance which consists of compounds occurring in nature and obtained from natural products or through synthesis. The natural substance may preferably contain at least one, preferably at least two of the compounds as defined above as main ingredient and additionally other essential oil compounds as for example capsaicin, tannin or carvacrol.

One example of the mixture of essential oils is CRINA® piglets which is commercially available from DSM Nutritional Products (Switzerland).

Preferably, the feed composition according to the present invention comprises benzoic acid, derivatives or metabolites thereof, in combination with at least two of thymol, eugenol and piperine. More preferably, the feed composition according to the present invention comprises benzoic acid, derivatives or metabolites thereof, in combination with a mixture of thymol, eugenol and piperine. One example of the feed composition comprising benzoic acid in combination with a mixture of thymol, eugenol and piperine is VevoWin® which is commercially available from DSM Nutritional Products (Switzerland).

The composition in the present invention may be in form of a feed additive or an animal feed.

Benzoic acid or a derivative thereof may be provided in an amount of from 500 mg/kg to 5,000 mg/kg animal feed, preferably from 1,000 mg/kg to 4,750 mg/kg animal feed, and more preferably from 2,000 mg/kg to 3,000 mg/kg animal feed.

Each of the essential oil such as thymol, eugenol and piperine may be provided in an amount of from 0.1 mg to 20 mg per kg animal feed (ppm), preferably from 1 mg to 10 mg per kg animal feed.

Examples of particularly preferred dosages of the essential oils in animal feed are independently from each other in the following ranges:
thymol between 1 ppm and 20 ppm, preferably between 1 ppm and 10 ppm;
eugenol between 1 ppm and 5 ppm, for example 2 ppm;
piperine up to 1 ppm, preferably between 0.3 ppm and 0.5 ppm.

The feed composition according to the present invention includes optionally other chemical compounds, for example at least one compound found in plants, and selected from the following group, as, per kg of animal feed,
up to about 1 mg of propylidene, butylidene, phtalides, gingerol, and/or lavender oil;
up to about 2 mg of decalactones, undecalactones, dodecalactones, ionones, irone, resorcinol, eucalyptol, menthol, peppermint oil, and/or alpha-pinene;
up to about 3 mg of limonene, guajacol, anethol, linalool, and/or methyl dihydrojasmonate;
up to about 4 mg of carvacrol, propionic, acetic or butyric acid, rosemary oil, clove oil, geraniol, terpineol, and/or citronellol;
up to about 5 mg of amyl, benzyl salicylate, cinnamaldehyde, and/or vanilline, a plant polyphenol (tannin); and/or
and up to about 5 mg of a powder of turmeric or of an extract of curcuma.

All these additional compounds may be used in combination with an emulsifying surfactant which may be selected advantageously from those of a rather hydrophilic nature, for example among polyglycerol esters of fatty acids such as esterified ricinoleic acid or propylene glycol esters of fatty acids, saccharo-esters or saccharo-glycerides, polyethylene glycol, lecithins etc.

Apart from the above ingredients, the feed composition according to the present invention contains at least one fat soluble vitamin, and/or at least one water soluble vitamin, and/or at least one trace mineral, and/or at least one macro mineral.

Examples of fat soluble vitamins include but are not limited to vitamin A, vitamin D3, vitamin E, and vitamin K, e.g. vitamin K3.

Examples of water soluble vitamins include but are not limited to vitamin B₁₂, biotin and choline, vitamin B₁, vitamin B₂, vitamin B₆, niacin, folic acid and panthothenate, e.g. Ca-D-panthothenate.

Examples of trace minerals include but are not limited to manganese, zinc, iron, copper, iodine, selenium, and cobalt.

Examples of macro minerals include but are not limited to calcium, phosphorus and sodium.

The term “animal” in the present invention includes all animals including human. Examples of animals are non-ruminants, and ruminants. Ruminant animals include, for example, animals such as sheep, goat, and cattle, e.g. cow such as beef cattle and dairy cows. Preferably, the animal is a non-ruminant animal. Examples of non-ruminant animals include but are not limited to horses, rabbits, pig or swine (including but not limited to, piglets, growing pigs, and sows), poultry such as turkeys, ducks, chickens (including but not limited to broiler chicks, layers), goose and pigeon. Especially preferably, the animal is swine.

The present invention is further illustrated with reference to the following Examples.

### EXAMPLES

### Example 1

### Material and Methods

A total of 22, weaner piglets (˜4 weeks old) were sourced from a commercial supplier following confirmation that they were seronegative for antibodies to Streptococcus suis. On arrival on site the animals were weighed and blood sampled then allocated to two groups of five or six piglets per pen based on ranked body weight. Animal arrival was on Day −10. Each group was placed in a separate pen.

From Day −10 until the end of the study, the animals from two pens were provided with a basal diet (Control Group), and the other two pens were provided with the basal diet supplemented with 3 g/Kg feed of VevoWin® (Test Group). The composition of the basal diet is shown in Table 1.

TABLE 1
Composition of the basal diet

| Ingredients (%) | |
|---|---|
| Barley | 15.00 |
| Oats | 5.00 |
| Wheat | 30.00 |
| Soya, extruded 48% | 16.00 |
| Soya, full fat | 3.00 |
| Fish meal 68% | 5.00 |
| Dry skimmed milk | 3.00 |
| Whey | 14.44 |
| Soya oil | 5.00 |
| Limestone | 0.20 |
| Salt | 0.01 |
| Lysine HCl | 0.35 |
| Methionine | 0.20 |
| Threonine | 0.17 |
| Tryptophan | 0.03 |
| Potato protein | 1.25 |
| Monodical. phosphate | 0.77 |
| L.-Valine | 0.05 |
| Phytase | 0.04 |
| Premix | 0.50 |

### Management and Sampling

Feed intake was recorded daily (based on weight of feed remaining in each feed trough each day). On Day 0, all animals were weighed and a clinical observation was carried out, then all animals (n=22) were challenged with 1×10⁸ CFU of Streptococcus suis by the intravenous route. A further clinical observation was performed on Day 0 four hours (±1 hour) post-challenge.

### Results

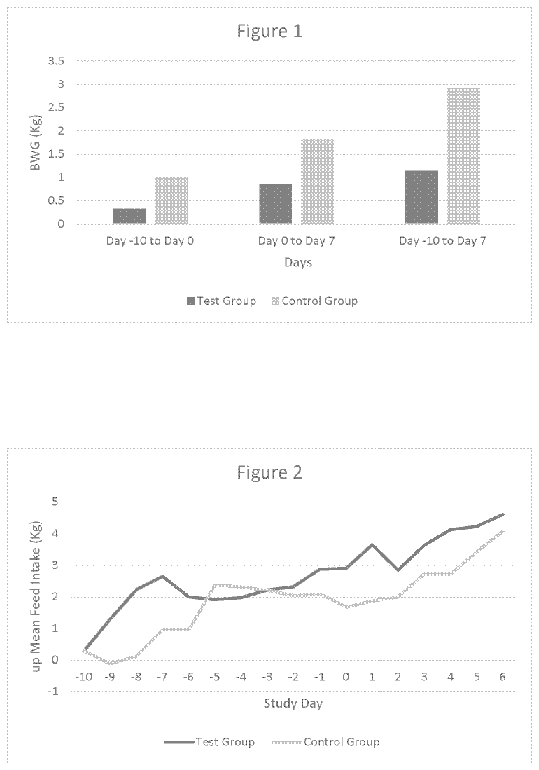

A summary of the mean bodyweight gain per piglet in each group is shown in FIG. 1. The data shown does not include bodyweights of animals euthanized between Day 2 and Day 6, although data for these animals on Day −10 and Day 0 is included.

As shown in FIG. 1, the bodyweight of both groups increased between arrival and Day 0 and Day 0 and Day 7. Compared to the Control Group, the animals in the Test Group showed higher increase in bodyweight between arrival and day 0, i.e., before the introduction of infection (1.02 vs 0.33 kg; p=0.0016), and between Day 0 and Day 7, i.e., after the introduction of infection.

A summary of the daily mean feed consumption per animal in each group is shown in FIG. 2. As shown in FIG. 2, feed intake from Day −10 up to day 0 (challenge) was higher in the Test Group than the Control Group; average feed intake for the total period was 2.26 kg/pig in Test Group compared to 1.49 kg/pig in Control Group.

### CONCLUSIONS

The feed additive, VevoWin®, provides positive effects on growth performance (bodyweight gain and feed intake) in the animals infected with Streptococcus suis.

1. A composition comprising benzoic acid, derivatives or metabolites thereof, in combination with at least one essential oil selected from the group consisting of thymol, eugenol and piperine, for improving growth performance of an animal infected with Streptococcus suis.

2. The composition of claim 1, comprising at least two, preferably at least three essential oils selected from the group consisting of thymol, eugenol and piperine.

3. The composition of claim 1, wherein the composition is in form of a feed additive or an animal feed.

4. The composition of claim 1, wherein benzoic acid, or a derivative or metabolite thereof, is provided in an amount of from 100 mg/kg to 5,000 mg/kg animal feed, preferably from 1,000 mg/kg to 4,750 mg/kg animal feed, and more preferably from 2,000 mg/kg to 3,000 mg/kg animal feed.

5. The composition of claim 1, wherein each of the essential oil is provided in an amount of from 0.1 mg to 20 mg per kg feed (ppm), preferably in the range of from 1 mg to 10 mg per kg animal feed.

6. The composition of claim 1, wherein the essential oil is provided independently from each other in the following ranges in animal feed:
thymol between 1 ppm and 20 ppm, preferably between 1 ppm and 10 ppm;
eugenol between 1 ppm and 5 ppm, for example 2 ppm; and
piperine up to 1 ppm, preferably between 0.3 ppm and 0.5 ppm.

7. The composition of claim 1, wherein the animal is a ruminant animal such as sheep, goat, and cattle, or a non-ruminant animal such as horse, rabbi, swine and poultry.

8. Use of the composition according to claim 1 for improving growth performance of an animal infected with Streptococcus suis.

9. A method for improving growth performance of an animal infected with Streptococcus suis, comprising administering to the animal an effective amount of the composition according to claim 1.
